(19) United States
(12) Patent Application Publication
(10) Pub. No.: US 2009/0318317 A1
(43) Pub. Date: Dec. 24, 2009

(54) LUBRICATING OIL FOR FLUID BEARING, AND FLUID BEARING AND METHOD FOR LUBRICATING FLUID BEARING BY USING THE LUBRICATING OIL

(75) Inventor: Yoshiyuki MORISHIMA, Toda-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: JAPAN ENERGY CORPORATION, Tokyo (JP); NIDEC CORPORATION, Kyoto (JP)

(21) Appl. No.: 12/440,148

(22) PCT Filed: Aug. 31, 2007
(86) PCT No.: PCT/JP2007/066972

§ 371 Date: Mar. 5, 2009

(30) Foreign Application Priority Data
Sep. 5, 2006 (JP) 2006-240362

(51) Int. Cl. C10M 133/44; C07C 69/66; C10M 105/36; C10M 129/18

(52) U.S. Cl. 508/100; 560/180; 508/497; 508/304; 508/279

(57) ABSTRACT

Disclosed is a lubricating oil for a fluid bearing, which has a low viscosity, a reduced amount of evaporation, and superior low-temperature flowability. The lubricating oil includes, as a base oil, a high-purity diester synthesized from a carboxylic acid material containing 90 mass % or more of azelaic acid and an alcohol material containing 90 mass % or more of 2-ethyl-1-hexanol. The carboxylic acid material contains glutaric acid, adipic acid, pimelic acid, suberic acid, sebacic acid and 1,9-nanomethylenedicarboxylic acid in a total amount of 5 mass % or less.

### FIELD OF THE INVENTION

The present invention relates to a lubricating oil for a fluid bearing, a fluid bearing using the lubricating oil and a method of lubricating the fluid bearing by using the lubricating oil; and, more particularly, to a lubricating oil for a fluid bearing, which has a low viscosity, a reduced amount of evaporation, and superior low-temperature flowability.

### BACKGROUND OF THE INVENTION

These days, electronic apparatuses including video/audio devices, personal computers and so on have advanced amazingly in terms of a reduction in size and weight and an increase in capacity and information processing speed. In the electronic apparatus, there is used a rotary device for driving a magnetic disk or an optical disk, such as a FD, a MO, a Zip, a mini disk, a compact disc (CD), a DVD, a hard disk or the like. Further, an improvement in a bearing essential for the rotary device greatly contributes to the reduction in the size and the weight of the electronic apparatus and the increase in the capacity and the processing speed thereof. Because a fluid bearing including a sleeve and a rotary shaft which are disposed to face each other with a lubricating oil filled therebetween does not employ a ball bearing, it is suitable for being used to reduce the size and the weight of the electronic apparatus. Also, the fluid bearing has superior quietness and generates economic benefits and thus is widely used in personal computers, sound devices, visual devices, car navigation systems and the like.

The lubricating oil for the fluid bearing is typically required to have lubricating properties, degradation stability (lifespan), resistance to sludge formation, wear resistance and corrosion resistance. With regard thereto, there have been proposed to date a lubricating oil composed of one or more selected from among olefin-, diester- and neopentylpolyolester-based synthetic oils, and squalane and naphthene-based mineral oil, and of a grease containing an urea compound as a thickener (see Japanese Patent Laid-open Publication No. Hei. 1-279117), a lubricating oil using trimethylolpropane fatty acid triester as a base oil and containing a hindered phenol-based oxidation inhibitor and a benzotriazole derivative (see Japanese Patent Laid-open Publication No. Hei. 1-188592), a lubricating oil containing a specific polymer-hindered phenol-based oxidation inhibitor and an aromatic amine-based oxidation inhibitor at a specified ratio (see Japanese Patent Laid-open Publication No. Hei. 1-225697), a lubricating oil using, as a base oil, specific monocarboxylic acid ester and/or specific dicarboxylic acid diester, having a phenyl group (see Japanese Patent Laid-open Publication No. Hei. 4-357318), a lubricating oil using a monomer composition as a base oil (see Japanese Patent No. 2621329), a lubricating oil using carbonate ester as a base oil and containing a sulfur-containing phenol-based oxidation inhibitor and a zinc-based extreme pressure additive (see Japanese Patent Laid-open Publication No. Hei. 8-34987), a lubricating oil containing a magnetic fluid (see Japanese Patent Laid-open Publication Nos. Hei. 8-259977, 8-259982, 8-259985), a lubricating oil using specific carbonate ester as a base oil and containing a phenol-based oxidation inhibitor (see Japanese Patent Laid-open Publication No. Hei. 10-183159), a lubricating oil containing trimethylolpropane and C4-8 monovalent fatty acid ester as a base oil (see Japanese Patent Laid-open Publication No. 2004-091524), a lubricating oil containing pimelic acid and/or suberic acid and a branched C6-10 monovalent alcohol diester as a base oil (see Japanese Patent Laid-open Publication No. 2004-250625), and a lubricating oil containing dicarboxylic acid and oxyalkylenealcohol diester as a base oil (see Japanese Patent Laid-open Publication No. 2006-096849).

Henceforth, the demand for high-speed processing of information of a large capacity or for reducing the size of the apparatus will be increase more and more. Conventionally, a sound device or a personal computer does not have a very large power consumption and is thus not receiving attention. Because attempts to reduce the size of an apparatus through use of a long lifespan or small capacity cell incorporated therein are made, wasted energy is still required to be further cut back on. Further, according to the demand for high-speed processing of information of a large capacity or for making reductions in the size of the apparatus, there is a need for higher-speed rotation of the fluid bearing. However, the loss of energy of the bearing increases proportionally to an increase in the speed. Many of the conventional lubricating oils for a fluid bearing have a high viscosity, therefore undesirably causing a great loss of energy of the bearing.

Further, the electronic apparatus is becoming widely popular, and is increasingly used under severe conditions. In particular, the apparatus mounted on the car, such as a car navigation system, should endure conditions of cold districts and those of hot weather in consideration of the environments in which the cars are used. Thus, a lubricating oil for a bearing used in the apparatus mounted on the car must be usable without problems in the wide temperature range of −40° C. to 80° C. For example, in the case where a lubricating oil having poor low-temperature flowability is used for a fluid bearing, it may solidify when used for a long period of time in a cold environment and thus the apparatus is rendered non-operational. Also, in the case where a lubricating oil having high evaporability is used for a fluid bearing, part of the lubricating oil may evaporate during use, making it impossible to sufficiently exhibit the lubricating function. Conventional lubricating oils for a fluid bearing are problematic in that an amount of evaporation is large when low-temperature flowability is superior, whereas low-temperature flowability is poor when an amount of evaporation is small, and thus low-temperature flowability and low evaporability are non-compatible with each other.

### SUMMARY OF THE INVENTION

Accordingly, the present invention has been made keeping in mind the problems encountered in the related art, and an aspect of the present invention is to provide a lubricating oil for a fluid bearing, which has a low viscosity, a reduced amount of evaporation and superior low-temperature flowability. In addition, another aspect of the present invention is to provide a fluid bearing using the lubricating oil and a method of lubricating the fluid bearing by using the lubricating oil.

Under a study completed for the purpose of accomplishing the above aspects, the present inventors discovered that a diester used as a base oil of a conventional lubricating oil for a fluid bearing may solidify upon extended storage at low temperature under a condition in which both the carboxylic acid-derived portion and the alcohol-derived portion of the diester are linear, and also that the viscosity thereof increases in the presence of a large amount of a high-molecular-weight component and the amount of evaporation thereof increases in the presence of a large amount of a low-molecular-weight component. Further, azelaic acid bis(2-ethylhexyl) synthesized from carboxylic acid, specifically azelaic acid, and an alcohol, specifically 2-ethyl-1-hexanol has been estimated to be optimal from the viewpoint of molecular structure and molecular weight. However, it has been found that the purity of azelaic acid in a conventional diester composed mainly of azelaic acid is very low to the level of about 70%, and low-temperature flowability and low evaporability are non-compatible with each other when such azelaic acid having a low purity is used as the material. Further, based on the aforementioned views, the present inventors have discovered that a high-purity diester containing azelaic acid bis(2-ethylhexyl) in a high concentration synthesized from high-purity materials can be used as the base oil of a lubricating oil for a fluid bearing, thus improving both low-temperature flowability and low evaporability of the lubricating oil while sufficiently lowering the viscosity of the lubricating oil, thereby completing the present invention.

Therefore, the present invention provides a lubricating oil for a fluid bearing, including, as a base oil, a high-purity diester synthesized from a carboxylic acid material containing 90 mass- or more of azelaic acid and an alcohol material containing 90 mass % or more of 2-ethyl-1-hexanol.

In the lubricating oil according to the present invention, the carboxylic acid material may contain glutaric acid, adipic acid, pimelic acid, suberic acid, sebacic acid and 1,9-nonamethylene dicarboxylic acid in a total amount of 5 mass % or less. The carboxylic acid material may contain glutaric acid, adipic acid and pimelic acid in a total amount of 3 mass % or less, and may contain 3 mass % or less of 1,9-nonamethylene dicarboxylic acid.

The lubricating oil according to the present invention preferably has a pour point of −50° C. or less and does not solidify even after storage at −40° C. for 30 days.

The lubricating oil according to the present invention may contain 95 mass % or more of the high-purity diester and 5 mass % or less of an additive. As the additive, an amine-based oxidation inhibitor may be contained in an amount of 0.01-5 mass %, and a phenol-based oxidation inhibitor may be contained in an amount of 0.1 mass % or less. Further, as the additive, at least one selected from the group consisting of an epoxy compound, a carbodiimide compound, and a triazole compound may be contained in an amount of 0.01-2 mass %.

In addition, the present invention provides a fluid bearing including a shaft, a sleeve, and the lubricating oil charged in a gap between the shaft and the sleeve. In addition, the present invention provides a method of lubricating the fluid bearing, including lubricating a gap between the shaft and the sleeve of the fluid bearing using the lubricating oil.

According to the present invention, the lubricating oil for a fluid bearing includes, as a base oil, high-purity diester synthesized from a carboxylic acid material containing 90 mass % or more of azelaic acid and an alcohol material containing 90 mass % or more of 2-ethyl-1-hexanol, and has a low viscosity, low-temperature flowability (after extended storage) and low evaporability. In addition, using such a lubricating oil, the fluid bearing and the method of lubricating the fluid bearing can be provided.

### BRIEF DESCRIPTION OF THE DRAWING

FIG. 1 is a schematic cross-sectional view showing a motor including a fluid bearing.

### DETAILED DESCRIPTION OF THE PREFERRED EMBODIMENTS

### Lubricating Oil for Fluid Bearing

Hereinafter, a lubricating oil for a fluid bearing in accordance with the present invention will be described in detail. The lubricating oil according to the present invention includes, as a base oil, a high-purity diester synthesized from a carboxylic acid material containing 90 masse or more of azelaic acid and an alcohol material containing 90 mass % or more of 2-ethyl-1-hexanol. The high-purity diester used as the base oil of the lubricating oil according to the present invention contains a large amount of azelaic acid bis(2-ethylhexyl), and a high-molecular-weight component or a low-molecular-weight component in an amount much smaller than the amount of azelaic acid bis(2-ethylhexyl). Because the high-purity diester has a low-molecular-weight component in an amount much smaller than the amount of azelaic acid bis(2-ethylhexyl), it has a reduced amount of evaporation. Further, azelaic acid bis(2-ethylhexyl) which is mainly contained in the high-purity diester has a branched alcohol-derived portion, and thus the diester has a low pour point and does not solidify even after extended storage at low temperature.

Used for the lubricating oil according to the present invention, the high-purity diester is synthesized through esterification between the carboxylic acid material containing 90 mass % or more of azelaic acid and the alcohol material containing 90 mass % or more of 2-ethyl-1-hexanol. If the amount of azelaic acid of the carboxylic acid material is less than 90 mass % and also the amount of 2-ethyl-1-hexanol of the alcohol material is less than 90 mass %, low-temperature flowability and low evaporability are not sufficiently compatible with each other.

Further, in the lubricating oil according to the present invention, azelaic acid bis(2-ethylhexyl) is used in an amount of 90 masse or more, and preferably 95 mass % or more. When the amount of azelaic acid bis(2-ethylhexyl) is 90 mass % or more, the lubricating oil may have a sufficiently low viscosity and also may have low-temperature flowability and low evaporability which are compatible with each other.

The carboxylic acid material may include glutaric acid, adipic acid, pimelic acid, suberic acid, sebacic acid and 1,9-nonamethylene dicarboxylic acid, in addition to azelaic acid. In this case, it is preferable that, in addition to azelaic acid, dicarboxylic acid species be contained in a total amount of 5 mass % or less in the carboxylic acid material. When the total amount of glutaric acid, adipic acid, pimelic acid, suberic acid, sebacic acid and 1,9-nonamethylene dicarboxylic acid is 5 mass % or less, the base oil for a lubricating oil having superior low-temperature flowability and sufficiently low evaporability can be synthesized.

Also, it is more preferable that, in the carboxylic acid material, glutaric acid, adipic acid and pimelic acid be contained in a total amount of 3 mass % or less. When the total amount of glutaric acid, adipic acid and pimelic acid in the carboxylic acid material is 3 mass % or less, low evaporability of the lubricating oil for a fluid bearing becomes particularly good.

Also, it is more preferable that, in the carboxylic acid material, 1,9-nonamethylene dicarboxylic acid may be contained in an amount of 3 mass % or less. When the amount of 1,9-nonamethylene dicarboxylic acid in the carboxylic acid material is 3 mass % or less, low-temperature flowability of the lubricating oil for a fluid bearing becomes particularly good.

On the other hand, the alcohol material may include 3,5,5-trimethyl-1-hexanol, in addition to 2-ethyl-1-hexanol. It is preferred that the amount of 2-ethyl-1-hexanol be 95 mass % or more.

The high-purity diester preferably has a dynamic viscosity at 40° C. of 10-11 mm²/s. When the dynamic viscosity at 40° C. of the above diester falls in the range of from 10 mm²/s to 11 mm²/s, the viscosity of the lubricating oil for a fluid bearing can be adequately lowered, thus achieving energy saving effects.

The lubricating oil according to the present invention may include 95 mass % or more of high-purity diester and 5 mass % or less of an additive. Examples of the additive include an amine-based oxidation inhibitor, a phenol-based oxidation inhibitor, an epoxy compound, a carbodiimide compound, and a triazole compound.

To the lubricating oil according to the present invention, the amine-based oxidation inhibitor may be added in an amount of 0.01-5 mass %, preferably 0.02-3 mass %, and more preferably 0.05-2 masse. When the amount of the amine-based oxidation inhibitor is 0.01 mass % or more, the lubricating oil can be adequately imparted with oxidation stability. Meanwhile, when the amount thereof is 5 mass % or less, the formation of sludge can be sufficiently inhibited.

Examples of the amine-based oxidation inhibitor include (1) monoalkyl diphenylamine such as monooctyl diphenylamine, monononyl diphenylamine or the like, (2) dialkyl diphenylamine such as 4,4′-dibutyl diphenylamine, 4,4′-dipentyl diphenylamine, 4,4′-dihexyl diphenylamine, 4,4′-diheptyl diphenylamine, 4,4′-dioctyl diphenylamine, 4,4′-dinonyl diphenylamine or the like, (3) polyalkyl diphenylamine such as tetrabutyl diphenylamine, tetrahexyl diphenylamine, tetraoctyl diphenylamine, tetranonyl diphenylamine or the like, (4) naphthylamine such as α-naphthylamine, phenyl-α-naphthylamine, butylphenyl-α-naphthylamine, pentylphenyl-α-naphthylamine, hexylphenyl-α-naphthylamine, heptylphenyl-α-naphthylamine, octylphenyl-α-naphthylamine, nonylphenyl-α-naphthylamine or the like, and derivatives thereof. Among them, dialkyl diphenylamine and alkylphenylnaphthylamine are preferable. Further, dialkyl diphenylamine and alkylphenylnaphthylamine, having a C4-24 alkyl group, are more preferable. Furthermore, dialkyl diphenylamine and alkylphenylnaphthylamine, having a C6-18 alkyl group, are still more preferable. The amine-based oxidation inhibitor species may be used alone or in combination of two or more.

The lubricating oil according to the present invention may include the phenol-based oxidation inhibitor, in addition to the amine-based oxidation inhibitor. In this case, the phenol-based oxidation inhibitor may be used in an amount of 0.1 mass % or less, preferably 0.03 masse or less, and more preferably 0.01 mass % or less. Most preferably, the phenol-based oxidation inhibitor is not used. When the amount of phenol-based oxidation inhibitor is 0.1 masse or less, the lubricating oil can be imparted with superior oxidation stability.

Examples of the phenol-based oxidation inhibitor include 2,6-di-t-butylphenol, 2,6-di-t-butyl-4-methylphenol, 4,4′-methylenebis(2,6-di-t-butylphenol), 4,4′-butylidenebis(3-methyl-6-t-butylphenol), 2,2′-methylenebis(4-ethyl-6-t-butylphenol), 2,2′-methylenebis(4-methyl-6-t-butylphenol), 4,4′-isopropylidenebisphenol, 2,4-dimethyl-6-t-butylphenol, tetrakis[methylene-3-(3,5-di-t-butyl-4-hydroxyphenyl)propionate]methane, 1,1,3-tris(2-methyl-4-hydroxy-5-t-butylphenyl)butane, 1,3,5-trimethyl-2,4,6-tris(3,5-di-t-butyl-4-hydroxybenzyl)benzene, 2,6-di-t-butyl-4-ethylphenol, 2,6-bis(2′-hydroxy-3′-t-butyl-5′-methylbenzyl)-4-methylphenol, bis[2-(2-hydroxy-5-methyl-3-t-butylbenzyl)-4-methyl-6-t-butylphenyl]terephthalate, triethyleneglycolbis[3-(3-t-butyl-5-methyl-4-hydroxyphenyl)propionate], 1,6-hexanediolbis[3-(3,5-di-t-butyl-4-hydroxyphenyl)propionate] and the like.

The lubricating oil according to the present invention may include, as an additive, 0.01-2 masse and preferably 0.02-1 mass % of at least one selected from among an epoxy compound, a carbodiimide compound, and a triazole compound. When the amount of this compound is 0.01 mass % or more, oxidation stability of the lubricating oil may be further improved and also hydrolysis stability may be improved. Meanwhile, when the amount thereof is 2 mass % or less, the formation of sludge may be sufficiently inhibited.

The epoxy compound has 4-60 carbons, and preferably 5-25 carbons. Specific examples of the epoxy compound include glycidylether such as butyl glycidylether, 2-ethylhexyl glycidylether, trimethylolpropane polyglycidylether, neopentylglycol diglycidylether, t-butylphenyl glycidylether or the like, glycidylester such as adipic acid glycidylester, 2-ethylhexanoic acid glycidylester, isononanoic acid glycidylester, neodecanoic acid glycidylester or the like, epoxylated fatty acid monoester such as epoxylated stearic acid methyl or the like, and epoxylated vegetable oil such as epoxylated soybean oil or the like. The epoxy compound may be glycidylether represented by Formula 1 below, glycidylether represented by Formula 2 below, or glycidylester represented by Formula 3 below.

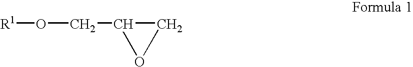

wherein R¹ is a hydrogen atom, a linear or branched C1-24 alkyl group, or a C7-24 alkylphenyl group.

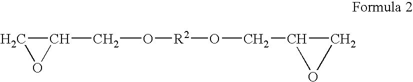

wherein R² is a linear or branched C1-18 alkylene group.

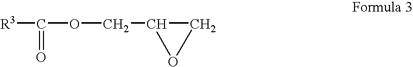

wherein R³ is a linear or branched C1-24 alkyl group, or a C7-24 alkylphenyl group. Particularly useful is glycidylether represented by Formula 1. The epoxy compound species may be used alone or in combination of two or more.

The carbodiimide compound may be represented by Formula 4 below.

R⁴—N═C═N—R⁵ Formula 4

wherein R4 and R5 are each independently a C1-24 hydrocarbon group, preferably a C7-24 alkylphenyl group and more preferably a C7-18 alkylphenyl group. Specific examples of the carbodiimide compound include 1,3-diisopropyl carbodiimide, 1,3-di-t-butyl carbodiimide, 1,3-dicyclohexyl carbodiimide, 1,3-di-p-tolyl carbodiimide, 1,3-bis(2,6-diisopropylphenyl)carbodiimide and the like. Among them, 1,3-diisopropyl carbodiimide, 1,3-di-p-tolyl carbodiimide and 1,3-bis(2,6-diisopropylphenyl) carbodiimide are preferable. The carbodiimide compound species may be used alone or in combination of two or more.

Examples of the triazole compound include benzotriazole and benzotriazole derivatives, and preferable examples thereof include a compound represented by Formula 5 below.

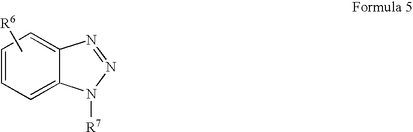

wherein R⁶ is a hydrogen atom or a methyl group and R⁷ is a hydrogen atom or a C0-20 monovalent group containing a nitrogen atom and/or an oxygen atom. As the triazole compound, a benzotriazole derivative is preferable, and a compound represented by Formula 5 in which R⁷ is a C5-20 monovalent group containing a nitrogen atom is more preferable. Specific examples of the triazole compound include 2-(2′-hydroxy-5′-methylphenyl)benzotriazole, 2-[2′-hydroxy-3′,5′-bis(α,α′-dimethylbenzyl)phenyl]benzotriazole, 2-(2′-hydroxy-3′,5′-di-t-butylphenyl)benzotriazole, 1-[N,N-bis(2-ethylhexyl)aminomethyl]benzotriazole and the like. The triazole compound species may be used alone or in a combination of two or more.

In addition, the lubricating oil according to the present invention may include a clear dispersant, an antiwear agent, a viscosity index improver, a pour point depressant, an ashless dispersant, a metal inactivator, a metallic cleanser, an oil agent, a surfactant, an antifoaming agent, a friction controller, a rust inhibitor, a corrosion inhibitor and the like, if necessary.

The lubricating oil according to the present invention has a pour point of −50° C. or less, from the viewpoint of low-temperature flowability. Also, the lubricating oil according to the present invention preferably does not solidify even after storage for 30 days at −40° C., from the viewpoint of low-temperature flowability after extended storage. Also, the lubricating oil according to the present invention preferably has a dynamic viscosity at 40° C. of 10-12 mm²/s and more preferably 10-11.5 mm²/s, from the viewpoint of energy saving effects.

The lubricating oil according to the present invention preferably has a total acid value of 1 mgKOH/g or less and more preferably 0.3 mgKOH/g or less, in terms of corrosion resistance, wear resistance and stability. Also, the lubricating oil according to the present invention preferably has a hydroxyl value of 20 mgKOH/g or less and more preferably 5 mgKOH/g or less, in terms of absorption resistance and stability. Also, the lubricating oil according to the present invention has a relative dielectric constant at 25° C. of 2.5 or more, preferably 2.7-10, and more preferably 2.9-8.0.

<Fluid Bearing and Method of Lubricating Fluid Bearing>

Next, the fluid bearing and the method of lubricating the fluid bearing according to the present invention will be specifically described. The fluid bearing according to the present invention includes a shaft, a sleeve, and the lubricating oil charged in a gap between the shaft and the sleeve. The method of lubricating the fluid bearing according to the present invention includes lubricating the gap between the shaft and the sleeve of the fluid bearing by using the lubricating oil. The fluid bearing according to the present invention is not mechanically limited as long as the sleeve and the shaft thereof are spaced apart from each other by a predetermined gap so that they do not come into direct contact with each other by means of the lubricating oil charged in the gap therebetween, without the interposition of a unit such as a ball bearing. The fluid bearing according to the present invention may include a fluid bearing which has dynamic pressure generating grooves formed in a rotary shaft and/or a sleeve and in which the rotary shaft is supported by dynamic pressure, or a fluid bearing having a thrust plate to generate dynamic pressure in a direction perpendicular to the rotary shaft.

The sleeve and the rotary shaft or the sleeve and the thrust plate are in partial or complete contact with each other in the absence of dynamic pressure upon non-rotation of the fluid bearing, while they are not in contact in the presence of dynamic pressure caused by rotation. In this way, when contact and non-contact are alternately repeated, the sleeve and the rotary shaft or the sleeve and the thrust plate may be subjected to metal wear, or sintering due to instant contact during rotation. However, when the lubricating oil according to the present invention having a low viscosity, a reduced amount of evaporation and superior low-temperature flowability is used, high-speed rotation stability and durability are maintained for a long period of time, and in particular, upon high-speed use, superior energy saving effects may be exhibited.

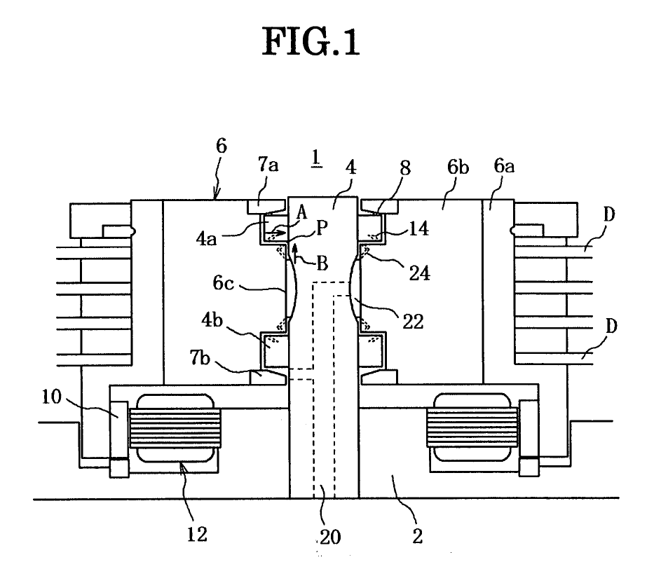

Below, with reference to the drawing, the fluid bearing and the method of lubricating the fluid bearing according to the present invention are described in detail. FIG. 1 is a schematic cross-sectional view showing a motor including a fluid bearing for driving a storage disk which uses the lubricating oil according to the present invention. As shown in FIG. 1, the motor 1 includes a bracket 2, a shaft 4 having one end fitted into the central opening of the bracket 2, and a rotor 6 relatively rotatably provided on the shaft 4. A rotational driving force is created between a stator 12 which is fixed to the bracket 2 and a rotor magnet 10 which is provided to the rotor 6 while facing the stator.

Also, at the upper and lower portions of the shaft 4, an upper thrust plate 4a and a lower thrust plate 4b each having a disk shape protruding radially outward are disposed. A gas receiving portion 22 is formed at the outer surface of the shaft between these thrust plates. The rotor 6 includes a rotor hub 6a having a storage disk D mounted on the outer peripheral surface thereof, and a sleeve 6b positioned on the inner periphery of the rotor 6 and supported to the shaft 4 by the small gap in which the lubricating oil 8 is charged. The sleeve 6b includes an upper counter plate 7a and a lower counter plate 7b covering the outer surfaces of the upper thrust plate and the lower thrust plate respectively.

As such, the small gap is defined between a region defined by the outer peripheral surface of the shaft 4 adjacent to the top of the gas receiving portion 22 at the center of the shaft 4 and the lower surface, the outer peripheral surface and the upper outer peripheral surface of the upper thrust plate 4a and a region defined by the top of the inner peripheral bore 6c of the sleeve 6b facing the shaft and the lower surface of the upper counter plate 7a, and is filled with the lubricating oil 8. Further, formed in the lower surface of the upper thrust plate 4a are spiral grooves 14 generating dynamic pressure of the lubricating oil 8 due to the rotation of the rotor 6. When the motor is rotated, a supporting force for maintaining the rotor in an axial direction is generated, and simultaneously, the lubricating oil 8 is thrust in a direction represented by the arrow A. Also formed in a lubricating oil-holding portion of the upper inner surface of the inner peripheral bore 6c of the sleeve 6b are unbalanced herringbone-shaped grooves 24. Upon rotation of the motor, a supporting force for maintaining the rotor in a radius direction is generated, and simultaneously, the lubricating oil 8 is thrust up in a direction represented by the arrow B.

The pressure distribution of the lubricating oil 8 in the small gap due to the dynamic pressure of the lubricating oil 8 caused by these grooves is highest in the lower inner periphery P of the lower surface of the upper thrust plate 4a. Thus, even when air dissolved in the lubricating oil 8 bubbles up, the air bubbles are prevented from diffusing to the outside of the inner periphery P, and reach the space of the gas receiving portion 22 or the space of the lower surface of the upper counter plate 7a. This space is directly exposed to the atmosphere or is exposed to the atmosphere by means of an air communication passage 20, and the air bubbles are exposed to the air, consequently realizing the fluid bearing having no leakage of the lubricating oil and having a high bearing capacity.

Likewise, the small gap, the grooves, and the lubricating oil-holding portion are disposed in reverse order over a region ranging from the bottom of the gas receiving portion 22 positioned at the center of the shaft 4 to the lower thrust plate 4b and the lower counter plate 7b. By means of the lower dynamic pressure bearing, the rotor may be more stably supported. Even when the fluid bearing according to the present invention is subjected to high-speed rotation of about 20,000 rpm, the outward diffusion of the lubricating oil 8 due to a rotational centrifugal force is effectively prevented by the upper and lower counter plates 7a, 7b. Further, when the fluid bearing according to the present invention is operated using the aforementioned lubricating oil, it can be used in the wide temperature range, can exhibit superior energy saving effects and durability, and can realize higher-speed and stable rotation.

### EXAMPLE

Below, a better understanding of the present invention may be obtained through the following examples, which are set forth to illustrate, but are not to be construed as limiting the present invention.

<High-Purity Diester>

A high-purity diester composed mainly of azelaic acid bis(2-ethylhexyl) was synthesized from a carboxylic acid material containing 99 mass % or more of azelaic acid and an alcohol material containing 99 mass % or more of 2-ethyl-1-hexanol. The high-purity diester thus obtained was analyzed through gas chromatography. As a result, the amount of azelaic acid bis(2-ethylhexyl) was measured to be greater than 99 mass %, and the other components were measured to be present only in trace amounts.

<Low-Purity Diester>

A low-purity diester composed mainly of azelaic acid bis(2-ethylhexyl) was synthesized from a carboxylic acid material containing 80 masse of azelaic acid and an alcohol material containing 99 mass % or more of 2-ethyl-1-hexanol. The low-purity diester thus obtained was analyzed through gas chromatography. As a result, the amount of glutaric acid bis(2-ethylhexyl) was measured to be 3.4 mass %, the amount of adipic acid bis(2-ethylhexyl) was measured to be 4.3 mass %, the amount of pimelic acid bis(2-ethylhexyl) was measured to be 4.9 masse, the amount of suberic acid bis(2-ethylhexyl) was measured to be 5.5 mass %, the amount of azelaic acid bis(2-ethylhexyl) was measured to be 73.0 masse, the amount of sebacic acid bis(2-ethylhexyl) was measured to be 3.3 masse, and the amount of 1,9-nonamethylene dicarboxylic acid bis(2-ethylhexyl) was measured to be 5.6 mass %.

<Evaluation of Base Oil>

A dynamic viscosity, an amount of evaporation, a pour point, low-temperature flowability, a total acid value, a hydroxyl value, and a relative dielectric constant of the high-purity diester and the low-purity diester were measured through the following methods. For comparison, azelaic acid di(n-octyl) and sebacic acid bis(2-ethylhexyl) were evaluated. The results are shown in Table 1 below.

(1) Dynamic Viscosity

A dynamic viscosity at 40° C. was measured using a Cannon-Fenske viscometer according to JIS K 2283.

(2) Amount of Evaporation

An amount of evaporation was determined from a mass decrement when allowing the diester to stand at 120° C. for 24 hours, through thermogravimetry (TG).

(3) Pour Point

A pour point was measured according to JIS K 2269.

(4) Low-Temperature Flowability

20 ml of a base oil for testing was placed in a 50 ml sample bottle and then allowed to stand at −40° C. for 30 days, and the flowability (solidification) of the base oil for testing was observed. After 30 days, when the sample bottle was turned upside down, a state in which the base oil therein did not flow within 1 min was classified as solidification.

(5) Total Acid Value

A total acid value was measured according to JIS K 2501.

(6) Hydroxyl Value

A hydroxyl value was measured according to JIS K 0070.

(7) Relative Dielectric Constant

A relative dielectric constant was measured at 25° C. according to JIS C 2101.

TABLE 1

| | Ex. 1 | C. Ex. 1 | C. Ex. 2 | C. Ex. 3 |
|---|---|---|---|---|
| Base Oil | High-purity | Low-purity | Azelaic Acid | Sebacic Acid |
| | diester | diester | Di(n-Octyl) | Bis(2-Ethylhexyl) |
| Dynamic | 10.4 | 10.4 | 10.7 | 11.6 |
| Viscosity at | | | | |
| 40° C. (mm²/s) | | | | |
| Amount of | 2.0 | 4.3 | 1.8 | 1.5 |
| Evaporation | | | | |
| (mass %) | | | | |
| Pour Point (° C.) | <−50 | <−50 | 10 | <−50 |
| Low-Temperature | Flowability | Flowability | Solidification | Flowability |
| Flowability | | | | |
| Total Acid Value | 0.01 | 0.01 | 0.01 | 0.01 |
| (mgKOH/g) | | | | |
| Hydroxyl Value | 1 | 1 | 1 | 2 |
| (mgKOH/g) | | | | |
| Relative | 4.1 | 4.1 | 4.1 | 3.9 |
| Dielectric | | | | |
| Constant at 25° C. | | | | |

As is apparent from Table 1, the high-purity diester of Example 1 synthesized from the carboxylic acid material containing 90 mass % or more of azelaic acid and the alcohol material containing 90 mass % or more of 2-ethyl-1-hexanol could be seen to have a low dynamic viscosity, a reduced amount of evaporation, and good low-temperature flowability. In contrast, the low-purity diester of Comparative Example 1 synthesized from the carboxylic acid material containing azelaic acid less than 90 mass % had an amount of evaporation greater than that of the diester of Example 1. Further, the azelaic acid di(n-octyl) of Comparative Example 2 had poor low-temperature flowability after extended storage. Also, sebacic acid bis(2-ethylhexyl) of Comparative Example 3 had a high dynamic viscosity and was poor in terms of energy saving effects.

<Evaluation of Oxidation Stability of Lubricating Oil for Fluid Bearing>

The base oil (high-purity diester) of Example 1 was mixed with additives shown in Table 2 below, thus preparing a lubricating oil for a fluid bearing, after which the oxidation stability of the lubricating oil thus obtained was evaluated through a rotary bomb oxidation test (RBOT) according to JIS K 2514. The results are shown in Table 2 below.

TABLE 2

| | Base | Additive (mass %) | | | | | | |
|---|---|---|---|---|---|---|---|---|
| | Oil | | Amine- | Amine- | | | | |
| | (mass %) | Phenol- | based | based | | | | Oxidation |
| | High- | based | Oxidation | Oxidation | | | | Stability |
| | Purity | Oxidation | Inhibitor | Inhibitor | Epoxy | Carbodiimide | Triazole | RBOT |
| | Diester | Inhibitor *1 | (DPA) *2 | (PNA) *3 | Compound *4 | Compound *5 | Compound *6 | (min) |
| Ex. 1 | 100.0 | — | — | — | — | — | — | 40 |
| Ex. 2 | 99.5 | 0.5 | — | — | — | — | — | 114 |
| Ex. 3 | 99.5 | — | 0.5 | — | — | — | — | 1897 |
| Ex. 4 | 99.5 | — | — | 0.5 | — | — | — | 1906 |
| Ex. 5 | 99.0 | 0.5 | 0.5 | — | — | — | — | 1278 |
| Ex. 6 | 99.0 | 0.5 | — | 0.5 | — | — | — | 804 |
| Ex. 7 | 99.4 | — | 0.5 | — | 0.1 | — | — | 2015 |
| Ex. 8 | 99.4 | — | 0.5 | — | — | 0.1 | — | 2260 |
| Ex. 9 | 99.4 | — | 0.5 | — | — | — | 0.1 | 2411 |
| Ex. 10 | 99.3 | — | 0.5 | — | 0.1 | — | 0.1 | 2557 |
| Ex. 11 | 99.2 | — | 0.5 | — | 0.1 | 0.1 | 0.1 | 2799 |
| Ex. 12 | 98.8 | 0.5 | 0.5 | — | 0.1 | — | 0.1 | 1408 |
| Ex. 13 | 98.8 | 0.5 | 0.5 | — | — | 0.1 | 0.1 | 1435 |
| Ex. 14 | 98.9 | 0.5 | 0.5 | — | — | — | 0.1 | 1666 |
| Ex. 15 | 98.7 | 0.5 | 0.5 | — | 0.1 | 0.1 | 0.1 | 1873 |

*1: 3-(3,5-di-t-butyl-4-hydroxyphenyl)propionic acid octyl, Irganox L135T available from Ciba Geigy Specialty Chemicals
*2: 4,4′-dioctyl diphenylamine, Vanlube 81 available from Vanderbilt
*3: octylphenyl-α-naphthylamine, Irganox L06 available from Ciba Geigy Specialty Chemicals
*4: 2-ethylhexyl glycidylether
*5: 1,3-bis(2,6-diisopropylphenyl) carbodiimide
*6: benzotriazole derivative, Irgamet 39 available from Ciba Geigy Specialty Chemicals

As is apparent from Table 2, the use of the amine-based oxidation inhibitor could be seen to further improve oxidation stability compared to when using the phenol-based oxidation inhibitor. When the amine-based oxidation inhibitor was used and the phenol-based oxidation inhibitor was not added, the improvement in oxidation stability was higher. Also, when the amine-based oxidation inhibitor was used and at least one selected from among an epoxy compound, a carbodiimide compound, and a triazole compound was further added, the oxidation stability of the lubricating oil was further improved.

### INDUSTRIAL APPLICABILITY

According to the present invention, the lubricating oil for a fluid bearing, and the fluid bearing using the lubricating oil and the method of lubricating the fluid bearing by using the lubricating oil can be applied not only to a motor including a fluid bearing for driving a storage disk which uses the lubricating oil but also to a rotary driving motor.

What is claimed is:

1. A lubricating oil for a fluid bearing, comprising, as a base oil, a high-purity diester synthesized from a carboxylic acid material containing 90 mass % or more of azelaic acid and an alcohol material containing 90 masse or more of 2-ethyl-1-hexanol.

2. The lubricating oil as set forth in claim 1, wherein the carboxylic acid material contains glutaric acid, adipic acid, pimelic acid, suberic acid, sebacic acid and 1,9-nonamethylene dicarboxylic acid in a total amount of 5 mass % or less.

3. The lubricating oil as set forth in claim 2, wherein the carboxylic acid material contains glutaric acid, adipic acid and pimelic acid in a total amount of 3 mass % or less.

4. The lubricating oil as set forth in claim 2, wherein the carboxylic acid material contains 3 mass % or less of 1,9-nonamethylene dicarboxylic acid.

5. The lubricating oil as set forth in claim 1, wherein the lubricating oil has a pour point of −50° C. or less and does not solidify even after storage at −40° C. for 30 days.

6. The lubricating oil as set forth in claim 1, wherein the lubricating oil contains 95 mass % or more of the high-purity diester and 5 mass % or less of an additive.

7. The lubricating oil as set forth in claim 6, wherein, as the additive, an amine-based oxidation inhibitor is contained in an amount of 0.01-5 mass %.

8. The lubricating oil as set forth in claim 7, wherein a phenol-based oxidation inhibitor is contained in an amount of 0.1 mass % or less.

9. The lubricating oil as set forth in claim 6, wherein, as the additive, at least one selected from the group consisting of an epoxy compound, a carbodiimide compound, and a triazole compound is contained in an amount of 0.01-2 mass %.

10. A fluid bearing, comprising a shaft, a sleeve, and the lubricating oil of any one of claims 1 to 9 charged in a gap between the shaft and the sleeve.

11. A method of lubricating a fluid bearing having a shaft and a sleeve, comprising lubricating a gap between the shaft and the sleeve by using the lubricating oil of any one of claims 1 to 9.
